(19) United States
(12) Patent Application Publication
(10) Pub. No.: US 2022/0328187 A1
(43) Pub. Date: Oct. 13, 2022

(54) CONDITION PREDICTING APPARATUS, CONDITION PREDICTING METHOD, COMPUTER PROGRAM, AND RECORDING MEDIUM

(71) Applicant: NEC Corporation, Minato-ku, Tokyo (JP)

(72) Inventors: Yuki Kosaka, Tokyo (JP); Toshinori Hosoi, Tokyo (JP); Masahiro Kubo, Tokyo (JP)

(73) Assignee: NEC Corporation, Minato-ku, Tokyo (JP)

(21) Appl. No.: 17/632,928

(22) PCT Filed: Aug. 9, 2019
(86) PCT No.: PCT/JP2019/031575

§ 371 Date: Feb. 4, 2022

(51) Int. Cl. G16H 50/20; G16H 10/60; G16H 50/70

(52) U.S. Cl. CPC G16H 50/20; G16H 10/60; G16H 50/70

(57) ABSTRACT

The condition predicting apparatus is provided with an acquisition unit configured to acquire target patient information indicating a condition of a target patient at a first time point of a hospitalization period, an extraction unit configured to extract, from a plural pieces of past patient information that respectively correspond to a plurality of other patients who are different from the target patient and that include first condition information indicating a condition at a time point corresponding to the first time point and second condition information indicating a condition at a time point corresponding to a second time point after the first time point of the hospitalization period of the target patient, one or more pieces of first past patient information that include the first condition information indicating a condition similar to the condition at the first time point indicated by the target patient information, an output unit configured to output at least a part of the one or more pieces of first past patient information, and a prediction unit configured to predict a condition of the target patient at the second time point based on the one or more pieces of first past patient information.

### TECHNICAL FIELD

The present invention relates to technical fields of a condition predicting apparatus, a condition predicting method, a computer program, and a recording medium.

### BACKGROUND ART

This type of apparatus may be used, for example, in the field of rehabilitation. For example, in Patent Reference 1, a technique is disclosed in which the number of rehabilitation sessions or the number of period units required for a target patient to advance to the next stage is predicted based on the clinical information of a plurality of patients in the past and the clinical information of the target patient, so that motivation of the target patient for rehabilitation is maintained and improved. Other related techniques are disclosed in Patent Literatures 2 to 5.

### CITATION LIST

### Patent Literature

### Patent Literature 1

Japanese Patent Laid-Open No. 2016-197330 A

### Patent Literature 2

Japanese Translation of PCT International Application Publication No. 2015-533433

### Patent Literature 3

Japanese Patent Laid-Open No. 2012-105795 A

### Patent Literature 4

Japanese Patent Laid-Open No. 2011-209885 A

### Patent Literature 5

International Publication No. WO 2018/030340

### SUMMARY

### Technical Problem

For example, a patient hospitalized to a convalescent rehabilitation hospital and a family member of the patient want to know how much functional recovery can be expected from rehabilitation. To address this situation, the condition of the patient at the time of discharge, for example, is predicted from the condition thereof at the time of hospitalization. However, there is also a technical problem that it is difficult for a healthcare worker, for example, to explain the predicted result to the patient and the family member of the patient in an easy-to-understand manner. The techniques described in the above Patent Literatures cannot solve this problem.

The present invention has been made in view of the above circumstances, and an objective of the present invention is to provide a condition predicting apparatus, a condition predicting method, a computer program, and a recording medium capable of predicting the degree of functional recovery made by rehabilitation with relatively high accuracy and outputting the predicted result in a manner that is easy for a user to understand.

### Solution to Problem

One example aspect of a condition predicting apparatus of the present invention includes: an acquisition unit configured to acquire target patient information indicating a condition of a target patient at a first time point of a hospitalization period; an extraction unit configured to extract, from a plural pieces of past patient information that respectively correspond to a plurality of other patients who are different from the target patient and that include first condition information indicating a condition at a time point corresponding to the first time point and second condition information indicating a condition at a time point corresponding to a second time point after the first time point of the hospitalization period of the target patient, one or more pieces of first past patient information that include the first condition information indicating a condition similar to the condition at the first time point indicated by the target patient information; an output unit configured to output at least a part of the one or more pieces of first past patient information; and a prediction unit configured to predict a condition of the target patient at the second time point based on the one or more pieces of first past patient information.

One example aspect of a condition predicting method includes: acquiring target patient information indicating a condition of a target patient at a first time point of a hospitalization period; extracting, from a plural pieces of past patient information that respectively correspond to a plurality of other patients who are different from the target patient and that include first condition information indicating a condition at a time point corresponding to the first time point and second condition information indicating a condition at a time point corresponding to a second time point after the first time point of the hospitalization period of the target patient, one or more pieces of first past patient information that include the first condition information indicating a condition similar to the condition at the first time point indicated by the target patient information; outputting at least a part of the one or more pieces of first past patient information; and predicting a condition of the target patient at the second time point based on the one or more pieces of first past patient information

One example aspect of a computer program causes a computer to execute one example aspect of the condition predicting method described above.

One example aspect of a recording medium is a recording medium in which one example aspect of the computer program described above is recorded.

### Advantageous Effect of Invention

According to one example aspect of each of the condition predicting apparatus, the condition predicting method, the computer program, and the recording medium described above, it is possible to output the predicted result in a manner that is easy for the user to understand.

### BRIEF DESCRIPTION OF THE DRAWINGS

FIG. 1 is a block diagram showing a hardware configuration of a condition predicting apparatus according to an example embodiment.

FIG. 2 is a block diagram showing a functional block realized in a CPU according to the example embodiment.

FIG. 3 is an example of a table showing observation data.

FIG. 4 is a first flowchart showing an operation of the condition predicting apparatus according to the example embodiment.

FIG. 5 is a second flowchart showing the operation of the condition predicting apparatus according to the example embodiment.

FIG. 6 is an example of an image presented to a user of the condition predicting apparatus according to the example embodiment.

FIG. 7 is a conceptual diagram showing a concept of grouping of past patients.

### DESCRIPTION OF EXAMPLE EMBODIMENT

An example embodiment of a condition predicting apparatus, a condition predicting method, a computer program, and a recording medium will be described based on drawings. Hereinafter, an example embodiment of the condition predicting apparatus, the condition predicting method, the computer program, and the recording medium will be described by using a condition predicting apparatus 1 that predicts at least one of a condition at the time of discharge and the number of hospitalization days of a patient in a convalescent rehabilitation hospital.

Here, in the convalescent rehabilitation hospital, an FIM (Functional Independence Measure) is often used as an index for measuring the effect of rehabilitation. Further, in the convalescent rehabilitation hospital, the condition at the time of discharge and the hospitalization period of the patient are often predicted, for example, based on the FIM at the time of hospitalization of the patient. However, accuracy and reliability of the prediction are not high. In addition, once the above prediction is made at the time of hospitalization of the patient, the above prediction is not to be made again. This may result in a deviation of the condition at the time of discharge and the hospitalization period, which have been predicted, from the actual condition at the time of discharge and the actual hospitalization period.

Therefore, in the condition predicting apparatus 1, FIMs (for example, an FIM at the time of hospitalization and an FIM at the time of discharge) of a patient, who has been discharged before one patient is hospitalized, are used to predict a condition and a hospitalization period of the one patient at a point of time after hospitalization (for example, at the time of discharge). Note that the “FIM at the time of hospitalization” is not limited to an FIM at a time point at which the patient is hospitalized, but is a concept that includes FIMs that can be practically regarded as the “FIMs at the time of hospitalization”, for example, such as an FIM obtained for the first time 1 to 2 days after the hospitalization date, and an FIM obtained before a time point at which the patient is hospitalized on the day of hospitalization. In the same manner, the “FIM at the time of discharge” is not limited to an FIM at a time point at which the patient is discharged, but is a concept that includes FIMs that can be practically regarded as the “FIMs at the time discharge”, for example, such as an FIM obtained at a time point at which the discharge date is specifically determined.

An outline of the condition predicting apparatus 1 will be described. The condition predicting apparatus 1 acquires target patient information indicating a condition of a target patient at a first time point of a hospitalization period. Here, the condition predicting apparatus 1 has a plural pieces of past patient information that respectively correspond to a plurality of other patients who are different from the target patient and that include first condition information (for example, an FIM at the time of hospitalization) indicating a condition at a time point corresponding to the above first time point and second condition information (for example, an FIM at the time of discharge) indicating a condition at a second time point after the above first time point of the hospitalization period of the target patient. The condition predicting apparatus 1 extracts, from the plural pieces of past patient information, one or more pieces of first past patient information that include the first condition information indicating a condition similar to the condition at the first time point indicated by the target patient information. Then, the condition predicting apparatus 1 predicts a condition of the target patient at the second time point based on the one or more pieces of first past patient information. The condition predicting apparatus 1 also predicts a hospitalization period of the target patient based on hospitalization periods of the one or more other patients corresponding to the one or more pieces of first past patient information. At this time, the condition predicting apparatus 1 outputs at least a part of the extracted one or more pieces of first past patient information (for example, presents it to a user of the condition predicting apparatus 1) in addition to the condition at the second time point and the hospitalization period that have been predicted.

Note that the “first time point” is not limited to the time at which the target patient is hospitalized, but may be any time point during the hospitalization period of the target patient, for example, one week after the hospitalization or the like. The “second time point” is not limited to the time at which the target patient is discharged, but may be any time point after the first time point during the hospitalization period of the target patient. With respect to the other patient, the “time point corresponding to the first (or second) time point” is one time point during the hospitalization period of the other patient, and means a time point corresponding to the first (or second) time point of the hospitalization period of the target patient. Specifically, for example, if the first (or second) time point is one week after the hospitalization, the “time point corresponding to the first (or second) time point” means one week after the hospitalization during the hospitalization period of the other patient.

Each of the plural pieces of past patient information described above includes progress information indicating a condition of the other patient observed at least at one time point from a time point corresponding to the above first time point to a time point corresponding to the above second time point in the hospitalization period of the other patient. After the condition and the hospitalization period of the target patient at the second time point are predicted, the condition predicting apparatus 1 acquires target patient progress information indicating a condition of the target patient observed after the above first time point and before the above second time point in the hospitalization period of the target patient. After that, the condition predicting apparatus 1 extracts, from the plural pieces of past patient information, one or more pieces of second past patient information that include the progress information indicating a condition similar to the condition indicated by the target patient progress information. Then, the condition predicting apparatus 1 corrects the condition at the second time point and the hospitalization period of the target patient, which have been predicted, based on the above one or more pieces of first past patient information and the above one or more pieces of second past patient information.

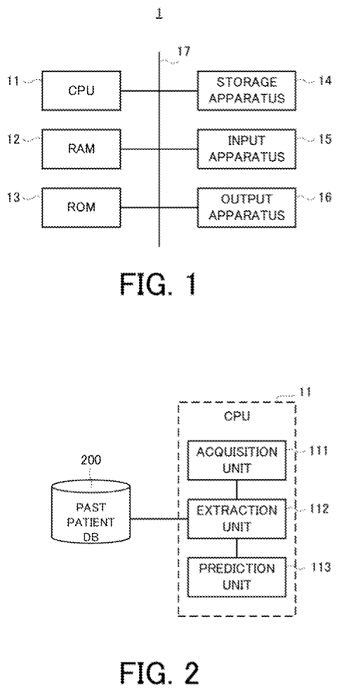

The example embodiment according to the condition predicting apparatus 1 will be specifically described with reference to FIGS. 1 to 7. In the example embodiment described below, a case is taken as an example in which the “first time point” and the “second time point” are “the time of hospitalization” and “the time of discharge”, respectively. Note that as described above, the “first time point” and the “second time point” are not limited to the “time of hospitalization” and the “time of discharge”, respectively.

First, a hardware configuration of the condition predicting apparatus 1 according to the example embodiment will be described with reference to FIG. 1. FIG. 1 is a block diagram showing a hardware configuration of the condition predicting apparatus 1 according to the example embodiment.

In FIG. 1, the condition predicting apparatus 1 is provided with a CPU (Central Processing Unit) 11, a RAM (Random Access Memory) 12, a ROM (Read Only Memory) 13, a storage apparatus 14, an input apparatus 15, and an output apparatus 16. The CPU 11, the RAM 12, the ROM 13, the storage apparatus 14, the input apparatus 15, and the output apparatus 16 are connected to each other via a data bus 17.

Note that the condition predicting apparatus 1 may be configured as a cloud system. In this case, the input apparatus 15 may have a configuration corresponding to a cloud system capable of receiving signals, information, and the like from an apparatus different from the condition predicting apparatus 1 such as a smartphone, a tablet, and a personal computer, for example. In the same manner, the output apparatus 16 may have a configuration corresponding to a cloud system capable of outputting signals, information, and the like to an apparatus different from the condition predicting apparatus 1.

The CPU 11 reads a computer program. For example, the CPU 11 may read a computer program stored in at least one of the RAM 12, the ROM 13, and the storage apparatus 14. For example, the CPU 11 may read a computer program stored in a computer-readable recording medium by using an unillustrated recording medium reading apparatus. The CPU 11 may acquire (that is, may read) a computer program, via a network interface, from an unillustrated apparatus arranged outside the condition predicting apparatus 1. The CPU 11 controls the RAM 12, the storage apparatus 14, the input apparatus 15, and the output apparatus 16 by executing the read computer program. Particularly, in the present example embodiment, when the CPU 11 executes the read computer program, a logical functional block for predicting at least one of a condition at the time of discharge and a hospitalization period of a patient is realized in the CPU 11. In other words, the CPU 11 can function as a controller for predicting at least one of the condition at the time of discharge and the hospitalization period of the patient. Note that the configuration of the functional block realized in the CPU 11 will be described in detail later with reference to FIG. 2.

The RAM 12 temporarily stores the computer program executed by the CPU 11. The RAM 12 temporarily stores data temporarily used by the CPU 11 when the CPU 11 executes the computer program. The RAM 12 may be, for example, a D-RAM (Dynamic RAM).

The ROM 13 stores the computer program executed by the CPU 11. In addition, the ROM 13 may also store fixed data. The ROM 13 may be, for example, a P-ROM (Programmable ROM).

The storage apparatus 14 stores data stored in the condition predicting apparatus 1 for a long period of time. The storage apparatus 14 may operate as a temporary storage apparatus of the CPU 11. The storage apparatus 14 may include, for example, at least one of a hard disk apparatus, a magneto-optical disk apparatus, an SSD (Solid State Drive), and a disk array apparatus.

The input apparatus 15 is an apparatus for receiving an input instruction from a user of the condition predicting apparatus 1. The input apparatus 15 may include, for example, at least one of a keyboard, a mouse, and a touch panel.

The output apparatus 16 is an apparatus for outputting information of the condition predicting apparatus 1 to the outside. For example, the output apparatus 16 may be a display apparatus capable of displaying information on the condition predicting apparatus 1.

Next, the configuration of the functional block realized in the CPU 11 will be described with reference to FIG. 2. FIG. 2 is a block diagram showing the functional block realized in the CPU 11.

As shown in FIG. 2, an acquisition unit 111, an extraction unit 112, and a prediction unit 113 are realized in the CPU 11 as logical functional blocks for predicting at least one of the condition at the time of discharge and the hospitalization period of the target patient.

In a past patient database 200 (hereinafter referred to as the “past patient DB 200” as appropriate), an FIM at the time of hospitalization and an FIM at the time of discharge of a patient who has been hospitalized to a convalescent rehabilitation hospital in the past (hereinafter referred to as the “past patient” as appropriate) are stored. Specifically, the FIM at the time of hospitalization and the FIM at the time of discharge are stored for each patient in association with patient condition information that indicates the condition of each patient.

Here, the patient condition information includes, for example, a gender, an age (or a generation), a family structure, a medical condition, a hospitalization date and a discharge date (or a hospitalization period), and the like. The past patient DB 200 as described above may be configured based on, for example, information of an electronic medical record. Note that the past patient DB 200 may be included in the condition predicting apparatus 1, or may be included in an apparatus different from the condition predicting apparatus 1.

Now, in the convalescent rehabilitation hospital, in order to observe the daily progress of patients, one or more observation items are often evaluated aside from the FIM. Examples of the observation items include “whether the patient was able to eat without a caregiver”, “whether the patient was able to change clothes without a caregiver”, “whether the patient was able to go to a toilet without a caregiver”, and the like. Examples of the evaluation include “was able”, “was partially able”, and “was not able”.

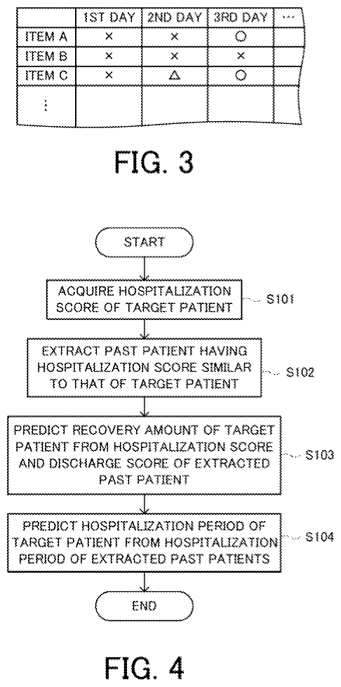

Information of the progress observation of the patient as described above is referred to as “observation data” in the present example embodiment. When the observation data is visually expressed, the observation data can be represented as a table as shown in FIG. 3, for example. (Note that in FIG. 3, “◯” represents “was able”, “Δ” represents “was partially able”, and “x” represents “was not able.”) The observation data is also stored for each patient in the past patient DB 200 in association with the patient condition information. Note that in addition to the evaluation (for example, “was able”, “was partially able”, “was not able”, and the like.), the observation data may include the contents of the implemented rehabilitation (for example, “walking training implemented”, “eating training implemented”, “stair training implemented”, and the like).

### (Operation)

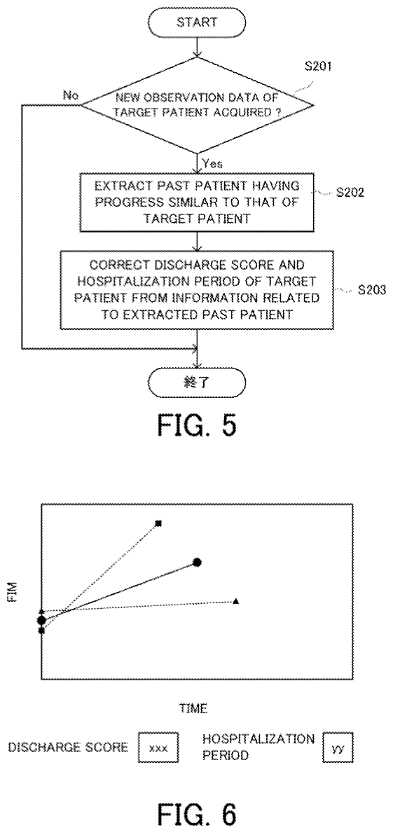

Operation of the condition predicting apparatus 1 will be described with reference to the flowcharts of FIGS. 4 and 5. The processing shown in the flowchart of FIG. 4 is performed when the FIM at the time of hospitalization of the target patient is obtained. On the other hand, the processing shown in the flowchart of FIG. 5 is performed during the hospitalization period of the target patient.

Here, in the condition predicting apparatus 1, a discharge score y_it, which is an FIM value at the time of discharge of the target patient, is predicted by adding a “recovery portion_it”, which is obtained by converting an estimated recovery amount predicted at time t into an FIM value, to a hospitalization score y_i_0, which is an FIM value at the time of hospitalization of the target patient. That is, “y_it=recovery portion_it+y_i_0”. Note that the suffix “i” is an identifier for identifying the target patient. The suffix “t” of “y_it” indicates that it is a predicted value at time t. In the following description, how to obtain the “recovery portion_it” will be mainly described.

In FIG. 4, first, the acquisition unit 111 acquires the hospitalization score, which is the FIM value at the time of hospitalization of the target patient (step S101). The hospitalization score of the target patient may be obtained from, for example, an electronic medical record system (not shown), or may be obtained from data input to the condition predicting apparatus 1 by the healthcare worker.

Next, the extraction unit 112 extracts, from the past patient DB 200, a past patient having a hospitalization score similar to that of the target patient (step S102). Specifically, for example, the extraction unit 112 first extracts, from the past patient DB 200, a past patient having patient condition information similar to the patient condition information of the target patient. This is because when the patient condition information of the past patient is similar to the patient condition information of the target patient, it is relatively likely that the contents of rehabilitation, for example, are similar, and thus it is relatively likely that the target patient follows the recovery process similar to that of the past patient After that, the extraction unit 112 extracts, from the extracted past patients, a past patient having the hospitalization score (that is, the FIM value at the time of hospitalization) similar to the hospitalization score of the target patient.

Next, the prediction unit 113 predicts a discharge score as one example that indicates a condition at the time of discharge of the target patient, from the hospitalization score of the past patient extracted in the processing of step S102 and the discharge score thereof which is the FIM value at the time of discharge (step S103).

Specifically, for example, the prediction unit 113 uses the following expression (1) to obtain an estimated recovery amount “recovery portion_i0” of the target patient at the time of hospitalization (that is, “time t=0”). In the expression (1), “xᵢ” is the hospitalization score and the patient condition information of the target patient i, and “xⱼ” is the hospitalization score and the patient condition information of the past patient j. “yⱼ” is the difference between the discharge score and the hospitalization score of the past patient j (that is, “discharge score−hospitalization score”). “s(xᵢ, xⱼ)” is the degree of similarity between the hospitalization score of the target patient i and the hospitalization score of the past patient j. “N” is the total number of past patients extracted in the processing of step S102.

$$\begin{matrix}[ Expression 1 ] & \\ recovery portion\_ i 0 = \frac{{\sum }_{j ∈ N} s ( {x}_{i} , {x}_{j} ) {y}_{j}}{{\sum }_{j ∈ N} s ( {x}_{i} , {x}_{j} )} & ( 1 )\end{matrix}$$

The prediction unit 113 predicts a discharge score y_i0 of the target patient by adding the “recovery portion_i0” obtained by using the expression (1) to the hospitalization score y_i_0 of the target patient.

In parallel with, or before or after the processing of step S103, the prediction unit 113 predicts the hospitalization period of the target patient from the hospitalization period of the past patient extracted in the processing of step S102 (step S104). This is because it is relatively likely that the hospitalization period of the target patient will be similar to the hospitalization period of the past patient having the patient condition information and the hospitalization score similar to those of the target patient (that is, the past patient extracted in the processing of step S112). The prediction unit 113 may predict the hospitalization period of the target patient by obtaining, for example, a simple or weighted average value, a median value, or the like of the hospitalization periods of the extracted past patients.

Note that the hospitalization period of the past patient may be obtained from the patient condition information stored in the past patient DB 200. Specifically, when the patient condition information includes the hospitalization period, that hospitalization period may be used. When the patient condition information includes the hospitalization date and the discharge date, the hospitalization period may be obtained from a difference between the discharge date and the hospitalization date.

Specifically, for example, the prediction unit 113 uses the following expression (2) to obtain a hospitalization period “hospitalization period_i0” of the target patient predicted at the time of hospitalization (that is, “time t=0”). Here, since the initial value of the hospitalization period (that is, the value corresponding to the hospitalization score y_i_0 of the target patient) is “0”, the hospitalization period of the target patient is determined only by the “hospitalization period_i0”. In the expression (2), “Tⱼ” is the (actual) hospitalization period of the past patient j.

$$\begin{matrix}[ Expression 2 ] & \\ hospitalization period\_ i 0 = \frac{{\sum }_{j ∈ N} s ( {x}_{i} , {x}_{j} ) {T}_{j}}{{\sum }_{j ∈ N} s ( {x}_{i} , {x}_{j} )} & ( 2 )\end{matrix}$$

The discharge score and the hospitalization period of the target patient predicted in the processing of steps S103 and S104 are presented to the user (for example, the healthcare worker, the target patient, the family member of the target patient, or the like) by the output apparatus 16 (for example, refer to FIG. 6).

For example, in FIG. 6, a graph showing a change in the FIM value from the time of hospitalization to the time of discharge of the target patient (refer to black dots and a solid line in FIG. 6) is presented together with the discharge score and the hospitalization period. In the graph of FIG. 6, the line shown with black triangles and a dotted line is a line connecting the hospitalization score and the discharge score of the past patient having the lowest discharge score among the past patients extracted in the processing of step S102. In the graph of FIG. 6, the line shown with black squares and a dotted line is a line connecting the hospitalization score and the discharge score of the past patient having the highest discharge score among the past patients extracted in the processing of step S102. By showing the lines of the past patients together with the line of the target patient, the user can relatively easily recognize the degree of fluctuation (in other words, an error) of each of the discharge score and the hospitalization period that have been predicted.

The condition of the target patient changes from moment to moment during hospitalization. Therefore, the discharge score and the hospitalization period of the target patient predicted by the above-mentioned processing may be different from the actual discharge score and the hospitalization period of the target patient. Then, the target patient may not recover the function to the expected extent or may not be able to be discharged at the expected time. Therefore, in the condition predicting apparatus 1, the discharge score and the hospitalization period of the target patient predicted by the above-mentioned processing are corrected (that is, updated) by using the “observation data” sequentially collected during the hospitalization of the target patient.

Now, when the number of samples of the information on the past patients (for example, the FIM at the time of hospitalization, the FIM at the time of discharge, the observation data, and the like) stored in the past patient DB 200 is not sufficient, the above correction may not function as expected (for example, a predicted error becomes large). Particularly, in the condition predicting apparatus 1, the above correction is made by dividing the information of the past patients into groups and then performing weighting for each group, so that information of the past patients including observation data that is likely reliable greatly contributes to the prediction and information of the past patients including observational data that is unlikely reliable does not contribute to the prediction (the details thereof will be described later). With such a configuration, the effect of improving the prediction accuracy (that is, of allowing the above correction to function well) can be expected.

When new information is acquired after a lapse of time from a certain time point (for example, at the time of hospitalization), it is often the case that the prediction is made again or the predicted result at the certain time point is corrected. Such re-prediction or correction works well when the value to be predicted (for example, the amount of recovery) can be periodically acquired as the above new information. This is because when the amount of recovery on a relatively close day to the discharge date is known, for example, it can be predicted that the amount of recovery on the day of discharge will not significantly change from the amount of recovery on the relatively close day.

On the other hand, when it is only possible to acquire information (for example, the observation data) that is different from the value to be predicted (for example, the recovery amount), the above-described re-prediction or correction is difficult. Even in such a case, when a correction algorithm is designed, for example, such that the predicted error becomes smaller each time new information is acquired after a lapse of time, it can be expected, for example, that the user trusts the predicted result after correction.

Therefore, in the condition predicting apparatus 1, by successfully managing weighting at the time of correction (the details thereof will be described later), prediction accuracy at the time of the current correction is improved from that at the time of the previous correction, and the result at the time of the current correction does significantly change from the result of the previous correction.

Specifically, in FIG. 5 (that is, during the hospitalization period of the target patient), the extraction unit 112 determines whether or not new observation data of the target patient has been acquired by the acquisition unit 111 (step S201). When it is determined in the processing of step S201 that the new observation data has not been acquired (step S201: No), the processing is terminated. Then, after a lapse of a predetermined time, the processing of step S201 is performed again.

When it is determined in the processing of step S201 that the new observation data has been acquired (step S201: Yes), the extraction unit 112 extracts a past patient having a progress similar to that of the target patient obtained from the observation data of the target patient (Step S202). Specifically, for example, the extraction unit 112 extracts, from the past patient DB 200, past patients having the patient condition information similar to the patient condition information of the target patient. After that, the extraction unit 112 extracts, from among the extracted past patients, a past patient having the observation data indicating a progress similar to the progress of the target patient.

Here, the “past patient having the observation data indicating a progress similar to that of the target patient” includes (i) a past patient having a recovery range similar to the degree of recovery (hereinafter referred to as the “recovery range” as appropriate) of the target patient in the period from the time of hospitalization of the target patient to the time when the new observation data is acquired (that is, the present), and (ii) a past patient to whom a rehabilitation menu similar to the rehabilitation menu implemented on the target patient has been implemented.

Next, the prediction unit 113 corrects the discharge score and the hospitalization period of the target patient based on the hospitalization scores and the discharge scores of the respective past patients extracted in the processing of step S102 (see FIG. 4) described above and the respective past patients extracted in the processing of step S202 (step S203). At this time, in the same manner as the processing of step S104 described above, for example, a graph showing a change in the FIM value from the time of hospitalization to the time of discharge of the target patient, the discharge score and the hospitalization period of the target patient, and a graph connecting the hospitalization score and the discharge score of the past patient are presented together (see FIG. 6). Note that the past patient whose graph is presented is not limited to the past patient extracted in the processing of step S102 described above, and may be the past patient extracted in the processing of step S202.

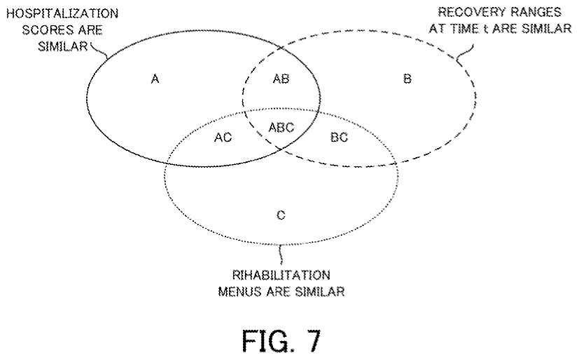

Specifically, the prediction unit 113 first classifies the past patients extracted in the processing of step S102 and the past patients extracted in the processing of step S202 into a plurality of groups (see, for example, FIG. 7). In FIG. 7, the past patients are classified into seven groups of a group A of the past patients who are similar to the target patient only in the hospitalization score, a group B of the past patients who are similar to the target patient only in the recovery range at time t, a group C of the past patients who are similar to the target patient only in the rehabilitation menu, a group AB of the past patients who are similar to the target patient in the hospitalization score and the recovery range at time t, a group AC of the past patients who are similar to the target patient in the hospitalization score and the rehabilitation menu, a group BC of the past patients who are similar to the target patient in the recovery range at time t and the rehabilitation menu, and a group ABC of the past patients who are similar to the target patient in the hospitalization score, the recovery range at time t, and the rehabilitation menu.

Here, the reason for grouping will be described. Ideally, when one or more patients who are almost or totally the same as the target patient are included in the past patients, the discharge score of the target patient can be accurately predicted, for example. However, in reality, such a past patient is unlikely to exist. Therefore, in the condition predicting apparatus 1, the past patients are grouped based on each piece of information (for example, the hospitalization score, the recovery range at time t, the rehabilitation menu, or the like) included in the information of the past patients (for example, the hospitalization score, one or more pieces of information included in the patient condition information, and the like). There may not be the past patient who is the same as the target patient in all items, but when attention is focused on one information (that is, the characteristics of the group), a past patient who is similar to the target patient is more likely to exist. That is, it is possible to increase the number of samples (that is, the number of past patients) that can be used when the discharge score and the like of the target patient are corrected. It is estimated that when the characteristics of the group are similar to those of the target patient, the discharge scores and the like of the past patients belonging to the group and the discharge score and the like of the target patient are also similar. As a result, improvement of the prediction accuracy can also be expected. For this reason, the past patients are classified (that is, grouped) as described above.

Next, by using the following expression (3), the prediction unit 113 obtains an estimated recovery amount “recovery portion_t” of the target patient at time t. In the expression (3), “x_{i,k}^{t}” is a hospitalization score of a target patient i and information including information that corresponds to a group k at time t (for example, the recovery range, the rehabilitation menus, and the like), and “x_{j,k}^{t}” is a hospitalization score of a past patient j included in the group k at time t and information including information that corresponds to the group k at time t. “yⱼ” is a difference between the discharge score and the hospitalization score of the past patient j (that is, “discharge score−the hospitalization score”). “s(x_{i,k}^{t}, x_{j,k}^{t})” is a degree of similarity between the target patient i and the past patient j included in the group k at time t. Here, for example, the degree of similarity may be obtained from the hospitalization score of the target patient i and the hospitalization score of the past patient j for the above group A, may be obtained from the recovery range of the target patient i and the recovery range of the target patient j for the above group B, and may be obtained from the rehabilitation menu of the target patient i and the rehabilitation menu of the past patient j for the above group C. For each of the groups AB, AC, BC and ABC, the degree of similarity may be obtained by using at least one of the hospitalization score, the recovery range, and the rehabilitation menu. “wₖ” is a weight related to the group k. “Nₖ^{t}” is a total number of past patients included in the group k at time t. “K” is a total number of groups (“K=7” when classified as shown in FIG. 7).

$$\begin{matrix}[ Expression 3 ] & \\ recovery portion\_ t = \frac{{\sum }_{k = 1}^{K} {\sum }_{j ∈ {N}_{k}^{t}} {w}_{k} s ( {x}_{i , k}^{t} , {x}_{j , k}^{t} ) {y}_{j}}{{\sum }_{k = 1}^{K} {\sum }_{j ∈ {N}_{k}^{t}} {w}_{k} s ( {x}_{i , k}^{t} , {x}_{j , k}^{t} )} & ( 3 )\end{matrix}$$

The prediction unit 113 predicts the discharge score y_it of the target patient by adding the “recovery portion_it” obtained by using the expression (3) to the hospitalization score y_i_0 of the target patient. The prediction unit 113 corrects the discharge score of the target patient by replacing a discharge score y_i(t−1) predicted at time t−1 with the discharge score y_it.

The prediction unit 113 also obtains a hospitalization period “hospitalization period_it” of the target patient predicted at time t by using the following expression (4). The prediction unit 113 corrects the hospitalization period of the target patient by replacing a hospitalization period_i(t−1) predicted at time t−1 with the hospitalization period_it. As described above, since the initial value of the hospitalization period (that is, the value corresponding to the hospitalization score y_i_0 of the target patient) is “0”, the hospitalization period of the target patient is determined only by the “hospitalization period_it”.

$$\begin{matrix}[ Expression 4 ] & \\ hospitalization period\_ i t = \frac{{\sum }_{k = 1}^{K} {\sum }_{j ∈ {N}_{k}^{t}} {w}_{k} s ( {x}_{i , k}^{t} , {x}_{j , k}^{t} ) {T}_{j}}{{\sum }_{k = 1}^{K} {\sum }_{j ∈ {N}_{k}^{t}} {w}_{k} s ( {x}_{i , k}^{t} , {x}_{j , k}^{t} )} & ( 4 )\end{matrix}$$

The discharge score and the hospitalization period of the target patient predicted in the processing of step S203 are presented to the user by the output apparatus 16 (see, for example, FIG. 6).

Here, the weight “wₖ” in the expressions (3) and (4) are obtained by using an expression that includes a regularization term so that, for example, the weight at time t−1 and the weight at time t are similar (in other words, so that there is no sudden change). Specifically, the weight “wₖ” is obtained by using the following expression (5).

$$\begin{matrix}[ Expression 5 ] & \\ {min}_{w} {\sum }_{t}^{T} {\sum }_{i}^{N} {( \hat{{y}_{ι}} - {y}_{i} )}^{2} + \frac{{λ}_{1}}{2} {‖ W ‖}_{F}^{2} + \frac{{λ}_{2}}{2} {\sum }_{t}^{T} {‖ {W}^{t} - {W}^{t - 1} ‖}_{F}^{2} & ( 5 )\end{matrix}$$

In the expression (5), “λ₁” and “λ₂” are regularization parameters. The first term represents a predicted error. The yᵢ hat in the first term represents a predicted value. Here, the predicted value corresponds to y_it=recovery portion_it+y_i_0, which is the predicted value obtained by the processing shown by the flowchart of FIG. 4. The second term is a term for reducing the weight w. In other words, the second term is a mechanism for enhancing a generalization capability to optimize the weight w. The regularization parameter λ₁ is a parameter for controlling how strongly the mechanism is considered to optimize the weight w. The larger λ₁ is, the more strongly the above mechanism is considered. The third term is a term for suppressing the sudden change in the weight w. The regularization parameter λ₂ is a parameter for controlling how strongly the sudden change of the weight w is considered to optimize the weight w. The larger λ₂ is, the more strongly the sudden change in the weight w is considered.

Since the term related to the temporal smoothing in the expression (5) is expressed as the following expression (6), the expression (5) can be expressed as the following expression (7). Here, the “term related to the temporal smoothing” is a “term for making the weight at time t−1 and the weight at time t similar (in other words, for preventing a sudden change)”. Note that in the expression (6), “H” is “H ¥in R^{{Tx(T-1)}}”, which means “H_{ij}=1 if i=j, H_{ij}=−1 if i=j+1, H_{ij}=0 otherwise”.

$$\begin{matrix}[ Expression 6 ] & \\ {\sum }_{t}^{T} {‖ {W}^{t} - {W}^{t - 1} ‖}_{F}^{2} = {‖ W H ‖}_{F}^{2} & ( 6 )\end{matrix}$$

$$\begin{matrix}[ Expression 7 ] & \\ {min}_{w} {\sum }_{t}^{T} {\sum }_{i}^{N} {( \hat{{y}_{ι}} - {y}_{i} )}^{2} + \frac{{λ}_{1}}{2} {‖ W ‖}_{F}^{2} + \frac{{λ}_{2}}{2} {‖ W H ‖}_{F}^{2} & ( 7 )\end{matrix}$$

Practically, it is desirable that as the time t increases (that is, as the number of hospitalization days of the target patient increases and the observation data is accumulated), the predicted error of the discharge score and the hospitalization period of the target patient becomes smaller. Therefore, it is desirable that the weight “wₖ” in the expressions (3) and (4) is determined by using the following expression (8).

$$\begin{matrix}[ Expression 8 ] & \\ {min}_{w} {\sum }_{t}^{T} {\sum }_{i}^{N} {( \hat{{y}_{ι}} - {y}_{i} )}^{2} + \frac{{λ}_{1}}{2} {‖ W ‖}_{F}^{2} + \frac{{λ}_{2}}{2} {‖ W H ‖}_{F}^{2} - {\sum }_{t}^{T} {\sum }_{i}^{N} σ \{ {( {\hat{y}}_{i , t - 1} - {y}_{i} )}^{2} - {( {\hat{y}}_{i , t} - {y}_{i} )}^{2} \} & ( 8 )\end{matrix}$$

In the expression (8), “σ” is a logit function and is expressed as “σ(α)=1/(1−exp^{-α})”. If α is 0 or larger, σ(α) is 0.5 or larger. Otherwise, σ(α) is less than 0.5.

### Technical Effect

According to the condition predicting apparatus 1 described above, as compared with a Comparative Example in which the condition of the target patient at the time of discharge and the like are predicted only from the condition of the target patient himself/herself at the time of hospitalization, for example, the degree of functional recovery by rehabilitation and the hospitalization period for the rehabilitation can be accurately predicted. Particularly, the condition predicting apparatus 1 can predict, at the time of hospitalization of the target patient, the degree of estimated functional recovery (for example, the discharge score) and the hospitalization period. Therefore, the target patient and the family member of the target patient are able to see, at the time of hospitalization, how much functional recovery can be expected by rehabilitation or how long the hospitalization period for the rehabilitation will be. This can reduce the anxiety of the target patient and the family member of the target patient. In addition, if the healthcare worker is able to know the estimated recovery of the function of the target patient, the healthcare worker can create a more appropriate implementation plan of rehabilitation.

In the condition predicting apparatus 1, as described with reference to FIG. 6, a graph connecting the hospitalization score and the discharge score of the past patient is presented to the user in addition to the degree of functional recovery and the hospitalization period of the target patient. Therefore, for example, as compared with a Comparative Example in which only a predicted result of the target patient is presented, the healthcare worker as a user can explain the result predicted by the condition predicting apparatus 1 to the target patient and the family member of the target patient in an easier-to-understand manner (for example, by comparing with the actual results of the past patients).

Further, in the condition predicting apparatus 1, when new observation data is acquired during the hospitalization period, the degree of estimated functional recovery and the hospitalization period of the target patient are corrected (that is, updated). Therefore, for example, if the healthcare worker refers to the degree of estimated functional recovery and the hospitalization period that have been corrected, the healthcare worker can appropriately determine whether the current implementation plan of rehabilitation is good or bad, appropriately determine whether or not to change the implementation plan, and the like.

As described with reference to FIG. 7, in the condition predicting apparatus 1, the past patients are first classified into groups and then the degree of similarity to the target patient “s(x_{i,k}^{t}, xⱼ, k^{t}) is obtained for each group. In addition, since the weight for calculating the “recovery amount_it” and the “hospitalization period_it” is obtained by using the expression (8), variation in prediction can be suppressed more as the observation data of the target patient is accumulated more after a lapse of time. Therefore, according to the condition predicting apparatus 1, the longer the hospitalization period of the target patient is (in other words, the closer it is to the discharge date of the target patient), the higher the reliability of the degree of estimated functional recovery and the hospitalization period, which have been predicted, are.

### <Modification>

The above-described “recovery portion_it” may also be obtained in the following manner. That is, from the hospitalization score, the discharge score, and the hospitalization period of each of the past patients extracted in the processing of step S102 (see FIG. 4) and/or the processing of step S202 (see FIG. 5), the prediction unit 113 obtains a slope of a recovery line of the each past patient (that is, “(discharge score −hospitalization score)/hospitalization period”). The prediction unit 113 may use the product of the average value of the obtained slopes and the average value of the hospitalization periods of the extracted past patients as the “recovery portion_it”. Note that the average value of the slopes and the average value of the hospitalization periods are desirably weighted average values weighted by the degree of similarity “s(xᵢ, xⱼ)” between the hospitalization score of the target patient i and the hospitalization score of the past patient j. The “recovery line” means a line passing the hospitalization score and the discharge score of one past patient (see, for example, FIG. 6).

<Supplementary Note>

With respect to the example example embodiments described above, the following Supplementary Notes will be further disclosed.

(Supplementary Note 1)

A condition predicting apparatus disclosed in a Supplementary Note 1 is a condition predicting apparatus, including:

an acquisition unit configured to acquire target patient information indicating a condition of a target patient at a first time point of a hospitalization period;

an extraction unit configured to extract, from a plural pieces of past patient information that respectively correspond to a plurality of other patients who are different from the target patient and that include first condition information indicating a condition at a time point corresponding to the first time point and second condition information indicating a condition at a time point corresponding to a second time point after the first time point of the hospitalization period of the target patient, one or more pieces of first past patient information that include the first condition information indicating a condition similar to the condition at the first time point indicated by the target patient information;

an output unit configured to output at least a part of the one or more pieces of first past patient information; and

a prediction unit configured to predict a condition of the target patient at the second time point based on the one or more pieces of first past patient information

(Supplementary Note 2)

A condition predicting apparatus disclosed in a Supplementary Note 2 is the condition predicting apparatus according to Supplementary Note 1, wherein the prediction unit predicts the condition of the target patient at the second time point based on the target patient information, and the first condition information and the second condition information included in the one or more pieces of first past patient information

(Supplementary Note 3)

A condition predicting apparatus disclosed in a Supplementary Note 3 is the condition predicting apparatus according to the Supplementary Note 1 or 2, wherein:

each of the plural pieces of past patient information includes progress information indicating a condition of one of the other patients observed at least at one time point from a time point corresponding to the first time point to a time point corresponding to the second time point;

the acquisition unit acquires target patient progress information indicating a condition of the target patient observed after the first time point and before the second time point;

the extraction unit extracts one or more pieces of second past patient information that include the progress information indicating a condition similar to the condition indicated by the target patient progress information; and

the prediction unit corrects the predicted condition of the target patient at the second time point based on the one or more pieces of first past patient information and the one or more pieces of second past patient information

(Supplementary Note 4)

A condition predicting apparatus disclosed in a Supplementary Note 4 is the condition predicting apparatus according to the Supplementary Note 3, wherein the prediction unit corrects the condition of the target patient at the second time point by predicting a recovery index value related to a degree of recovery of the target patient based on the first condition information and the second condition information included in the one or more pieces of first past patient information, and the first condition information and the second condition information included in the one or more pieces of second past patient information

(Supplementary Note 5)

A condition predicting apparatus disclosed in a Supplementary Note 5 is the condition predicting apparatus according to the Supplementary Note 4, wherein the prediction unit classifies the one or more pieces of first past patient information and the one or more pieces of second past patient information into a plurality of groups, and predicts the recovery index value based on a weight determined for each of the plurality of groups, the first condition information and the second condition information included in the one or more pieces of first past patient information, and the first condition information and the second condition information included in the one or more pieces of second past patient information

(Supplementary Note 6)

A condition predicting apparatus disclosed in a Supplementary Note 6 is the condition predicting apparatus according to any one of the Supplementary Notes 1 to 3, wherein the prediction unit corrects the condition of the target patient at the second time point so as to satisfy at least one of a requirement for which an amount of change is suppressed from a previous correction result to a current correction result and a requirement for which a predicted error becomes smaller as the target patient progress information is accumulated with a lapse of time after the first time point.

(Supplementary Note 7)

A condition predicting apparatus disclosed in a Supplementary Note 7 is the condition predicting apparatus according to the Supplementary Note 1, wherein:

the first time point is a time point when the target patient is hospitalized;

the second time point is a time point when the target patient is discharged; and

the prediction unit predicts a condition at a time when the target patient is discharged based on the one or more pieces of first past patient information

(Supplementary Note 8)

A condition predicting apparatus disclosed in a Supplementary Note 8 is the condition predicting apparatus according to the Supplementary Note 1, wherein the prediction unit predicts the hospitalization period of the target patient based on one or more hospitalization periods of the other patients corresponding to the one or more pieces of first past patient information, in addition to or instead of predicting the condition of the target patient at the second time point.

(Supplementary Note 9)

A condition predicting apparatus disclosed in a Supplementary Note 9 is the condition predicting apparatus according to Supplementary Note 8, wherein:

each of the plural pieces of past patient information includes progress information indicating a condition of one of the other patients observed at least at one time point from a time point corresponding to the first time point to a time point corresponding to the second time point;

the acquisition unit acquires target patient progress information indicating a condition of the target patient observed after the first time point and before the second time point;

the extraction unit extracts one or more pieces of second past patient information that include the progress information indicating a condition similar to the condition indicated by the target patient progress information; and

the prediction unit corrects the predicted hospitalization period of the target patient based on the one or more pieces of first past patient information and the one or more pieces of second past patient information

(Supplementary Note 10)

A condition predicting method disclosed in a Supplementary Note 10 is a condition predicting method, including:

acquiring target patient information indicating a condition of a target patient at a first time point of a hospitalization period;

extracting, from a plural pieces of past patient information that respectively correspond to a plurality of other patients who are different from the target patient and that include first condition information indicating a condition at a time point corresponding to the first time point and second condition information indicating a condition at a time point corresponding to a second time point after the first time point of the hospitalization period of the target patient, one or more pieces of first past patient information that include the first condition information indicating a condition similar to the condition at the first time point indicated by the target patient information;

outputting at least a part of the one or more pieces of first past patient information; and

predicting a condition of the target patient at the second time point based on the one or more pieces of first past patient information.

(Supplementary Note 11)

A computer program disclosed in a Supplementary Note 11 is a computer program that causes a computer to execute the condition predicting method according to the Supplementary Note 10.

(Supplementary Note 12)

A recording medium disclosed in a Supplementary Note 12 is a recording medium in which the computer program according to the Supplementary Note 11 is recorded.

(Supplementary Note 13)

A condition predicting apparatus disclosed in a Supplementary Note 13 is the condition predicting apparatus according to Supplementary Note 1, wherein:

the predicting unit predicts the condition of the target patient at the second time point from the condition at the first time point indicated by the target patient information and an index indicating a change in the condition obtained based on the first condition information and the second condition information included in the one or more pieces of first past patient information.

The present invention can be appropriately modified within the scope not contrary to the gist or idea of the invention that can be read from the claims and the entire specification, and a condition predicting apparatus, a condition predicting method, a computer program, and a recording medium accompanied by such a modification are also included in the technical idea of the present invention.

### BRIEF DESCRIPTION OF REFERENCE NUMBERS

1 Condition predicting apparatus
11 CPU
111 Acquisition unit
112 Extraction unit
113 Prediction unit
200 Past patient DB

What is claimed is:

1. A condition predicting apparatus, comprising:
at least one memory configured to store instructions; and
at least one processor configured to execute the instructions to:
acquire target patient information indicating a condition of a target patient at a first time point of a hospitalization period;
extract, from a plural pieces of past patient information that respectively correspond to a plurality of other patients who are different from the target patient and that include first condition information indicating a condition at a time point corresponding to the first time point and second condition information indicating a condition at a time point corresponding to a second time point after the first time point of the hospitalization period of the target patient, one or more pieces of first past patient information that include the first condition information indicating a condition similar to the condition at the first time point indicated by the target patient information;
output at least a part of the one or more pieces of first past patient information; and
predict a condition of the target patient at the second time point based on the one or more pieces of first past patient information.

2. The condition predicting apparatus according to claim 1, wherein the at least one processor configured to execute the instructions to predict the condition of the target patient at the second time point based on the target patient information, and the first condition information and the second condition information included in the one or more pieces of first past patient information.

3. The condition predicting apparatus according to claim 1, wherein:
each of the plural pieces of past patient information includes progress information indicating a condition of one of the other patients observed at least at one time point from a time point corresponding to the first time point to a time point corresponding to the second time point;
the at least one processor configured to execute the instructions to:
acquire target patient progress information indicating a condition of the target patient observed after the first time point and before the second time point;
extract one or more pieces of second past patient information that include the progress information indicating a condition similar to the condition indicated by the target patient progress information; and
correct the predicted condition of the target patient at the second time point based on the one or more pieces of first past patient information and the one or more pieces of second past patient information.

4. The condition predicting apparatus according to claim 3, wherein the at least one processor configured to execute the instructions to correct the condition of the target patient at the second time point by predicting a recovery index value related to a degree of recovery of the target patient based on the first condition information and the second condition information included in the one or more pieces of first past patient information, and the first condition information and the second condition information included in the one or more pieces of second past patient information.

5. The condition predicting apparatus according to claim 4, wherein the at least one processor configured to execute the instructions to classify the one or more pieces of first past patient information and the one or more pieces of second past patient information into a plurality of groups, and predict the recovery index value based on a weight determined for each of the plurality of groups, the first condition information and the second condition information included in the one or more pieces of first past patient information, and the first condition information and the second condition information included in the one or more pieces of second past patient information.

6. The condition predicting apparatus according to claim 3, wherein the at least one processor configured to execute the instructions to correct the condition of the target patient at the second time point so as to satisfy at least one of a requirement for which an amount of change is suppressed from a previous correction result to a current correction result and a requirement for which a predicted error becomes smaller as the target patient progress information is accumulated with a lapse of time after the first time point.

7. The condition predicting apparatus according to claim 1, wherein:
the first time point is a time point when the target patient is hospitalized;
the second time point is a time point when the target patient is discharged; and
the at least one processor configured to execute the instructions to predict a condition at a time when the target patient is discharged based on the one or more pieces of first past patient information.

8. The condition predicting apparatus according to claim 1, wherein the at least one processor configured to execute the instructions to predict the hospitalization period of the target patient based on one or more hospitalization periods of the other patients corresponding to the one or more pieces of first past patient information, in addition to or instead of predicting the condition of the target patient at the second time point.

9. The condition predicting apparatus according to claim 8, wherein:
each of the plural pieces of past patient information includes progress information indicating a condition of one of the other patients observed at least at one time point from a time point corresponding to the first time point to a time point corresponding to the second time point;
the at least one processor configured to execute the instructions to:
acquire target patient progress information indicating a condition of the target patient observed after the first time point and before the second time point;
extract one or more pieces of second past patient information that include the progress information indicating a condition similar to the condition indicated by the target patient progress information; and
correct the predicted hospitalization period of the target patient based on the one or more pieces of first past patient information and the one or more pieces of second past patient information.

10. A condition predicting method, comprising:
acquiring target patient information indicating a condition of a target patient at a first time point of a hospitalization period;
extracting, from a plural pieces of past patient information that respectively correspond to a plurality of other patients who are different from the target patient and that include first condition information indicating a condition at a time point corresponding to the first time point and second condition information indicating a condition at a time point corresponding to a second time point after the first time point of the hospitalization period of the target patient, one or more pieces of first past patient information that include the first condition information indicating a condition similar to the condition at the first time point indicated by the target patient information;
outputting at least a part of the one or more pieces of first past patient information; and
predicting a condition of the target patient at the second time point based on the one or more pieces of first past patient information.

11. (canceled)

12. A non-transitory recording medium in which a computer program that causes a computer to execute the condition predicting method according to claim 10 is recorded.
